(19) United States
(12) Patent Application Publication
(10) Pub. No.: US 2005/0170400 A1
(43) Pub. Date: Aug. 4, 2005

(54) Method for identifying bead location information

(75) Inventor: Keiichi Sato, Tokyo (JP)

Correspondence Address: Reed Smith LLP, Suite 1400, 3110 Fairview Park Drive, Falls Church, VA, 22042-4503, US

(73) Assignee: Hitachi Software Engineering Co., Ltd.

(21) Appl. No.: 11/041,263

(22) Filed: Jan. 25, 2005

(30) Foreign Application Priority Data
Feb. 2, 2004 (JP) 25890/2004

(51) Int. Cl. C12Q 1/68; G01N 33/543

(52) U.S. Cl. 435/6; 436/523

(57) ABSTRACT

Location information of carriers such as beads arranged inside a capillary represented by a probe array is detected accurately, and an identification method for various materials is developed substituting conventional bar codes for the beads. The arrangement order of a plurality of beads is identified on the basis of stain color information and location information concerning the plurality of stained beads.

### BACKGROUND OF THE INVENTION

1. Field of the Invention

The present invention relates to a method for identifying the order of beads arranged inside a capillary in probe array technology, for example, regarding biopolymer detection and measurement.

2. Background Art

In biopolymer detection, measurement, and analysis technologies (hereafter all referred to as “detection”), various methods have been proposed, not a few of which have already been manufactured for practical use. They include a method where well-known probe biopolymers are immobilized on a base and a substrate, and sample biopolymers labeled with fluorescent materials are then subjected to a reaction with the probe biopolymers immobilized on the aforementioned base or the substrate. The amount of bonding of the probe biopolymers and the sample biopolymers is measured using fluorescence intensity resulting from the fluorescent materials used for labeling on the base or the substrate, thereby allowing the biopolymers to be detected. The aforementioned substrate or base is generally constructed in the form of either a plate of a bead, and are attracting attention as a microarray technology and a technology using beads, respectively.

DNA microarray is a method in which the plate is used as the substrate or the base. The DNA microarray normally has probe DNA arranged as spots and immobilized on a surface-modified glass base. There are two main types of DNA microarray: a method where the probe DNA is synthesized on the base applying semiconductor technology, and a method where previously synthesized oligo DNA or cDNA is arranged and immobilized as droplets on the base.

Also, flow cytometry technology is a method in which the beads are used as the substrate or the base. In this method, the types of probes are identified by providing each bead with ID resulting from the use of two or more colors of fluorescent materials or pigments, as compared with the DNA microarray by which the types of immobilized probes are identified using location information.

The microarray technology as represented by the DNA microarray is widely used mainly for research, as it has a high degree of accumulation, and an enormous amount of information can be obtained by a single operation. However, it poses problems in that advanced skills are required in the process of immobilizing the probes, that production cost per plate is high, and that the bonding strength of the substrate or the base is relatively weak upon immobilization of the probes. By contrast, the bead technology is superior in that the bonding of the probes and the substrate or the base is relatively strong, and that the synthesis of beads can be performed at a relatively low price, although advanced skills are required upon providing ID for the beads. Thus, each technology has advantages and disadvantages. Additionally, while the methods in which DNA is used are described in the aforementioned examples, there has been technological progress in which DNA is substituted for peptides, proteins, or cells.

Thus, as disclosed in JP Patent Publication (Kokai) No. 2000-346842 A , for example, the probe array technology is devised as an integrated form of the microarray technology and the technology using beads. This technology is characterized by separating the steps of immobilizing the probes on the substrate or the base and of arranging the probes on the base. In other words, the probes are immobilized on the beads in advance, and then the beads are arranged in the channel of the base or inside a capillary. The types of the probes can be determined on the basis of the location information of the beads, and a reaction is conducted by allowing a sample solution to pass the channel or the capillary. By using this technology, many of the problems of the microarray technology and the technology using beads can be solved. Further, the reactivity of the probes and the sample can be improved and apparatus automation can be achieved relatively readily. However, the difficulty of conducting an examination to determine whether the order of beads is correct, for example, upon arranging the beads is problematic, since the beads are not provided with ID.

### SUMMARY OF THE INVENTION

It is an object of the present invention to provide technology that readily enables the detection of whether the order of beads arranged in the channel of a base or inside a capillary is correct, regarding probe array technology in which the microarray technology and the technology using beads are integrated.

In a first aspect, the present invention is a method for identifying the arrangement state of beads. The arrangement state of a plurality of beads is identified on the basis of the stain color information and the location information concerning the aforementioned plurality of types of stained beads. In this case, the aforementioned plurality of types of beads may be arranged in a single line in the channel of a bead array, the aforementioned location information may be one-dimensional, and the aforementioned arrangement state may be one-dimensional. Or, the aforementioned plurality of types of beads may be immobilized and arranged in a planar manner, the aforementioned location information may be two-dimensional, and the aforementioned arrangement state may be two-dimensional. Additionally, colorless (namely, unstained) beads are counted as one type among “the plurality of types of stained beads.” Thus, beads composed of unstained beads and one type of stained bead are included among “the plurality of types of stained beads” according to the present invention.

The wavelengths of the stain of the aforementioned plurality of beads are not limited, and so any wavelengths would be fine as long as ultraviolet rays, visible light, or infrared rays are emitted.

Preferably, the aforementioned plurality of stained beads are specifically beads that have embedded semiconductor nanoparticles, or beads that carry the semiconductor nanoparticles on the surface, and the aforementioned stain color is preferably fluorescent. Or, preferably, the aforementioned plurality of stained beads are beads that have embedded stain or pigment, or beads that carry the stain or the pigment on the surface, and the aforementioned stain color is a visible light color.

In a second aspect, the present invention provides a method for identifying the order of the beads arranged inside a capillary. In the probe array technology for detecting biopolymers in which a plurality of beads are arranged inside a capillary, the present invention uses an identification method for identifying the arrangement state of the aforementioned plurality of beads so as to identify the order of the beads arranged inside the capillary on the basis of the stain color information and location information concerning a plurality of stained beads.

The wavelengths of the emissions of the aforementioned plurality of stained beads are not limited, and so any wavelengths can be used as long as ultraviolet rays, visible light, or infrared rays are emitted. Specifically, the aforementioned plurality of stained beads are preferably beads that have embedded semiconductor nanoparticles, or beads that carry the semiconductor nanoparticles on the surface, and the aforementioned stain color is preferably fluorescent. Or, preferably, the aforementioned plurality of stained beads are beads that have embedded stain or pigment, or beads that carry the stain or the pigment on the surface, and the aforementioned stain color is a visible light color.

In the present invention, the beads are stained with various pigments, and then the order of the beads arranged in various materials, such as channels or capillaries, is identified. Although the pigments for staining the beads are preferably monochrome taking into consideration high distinction and macroscopic visibility, a plurality of colors can be used. Although the pigments for use can be conventional organic fluorescent pigments, for example, more preferably, semiconductor nanoparticles are used, since a multitude of fluorescent colors can be simultaneously excited by a single excitation light source. In other words, semiconductor nanoparticles, represented by CdS, CdSe, and the like, are embedded in the beads, and the arrangement order of the beads is identified. The present invention does not require as many pigment colors as the number of types of beads, and a number of colors that compliments merely the range of error is sufficient.

According to the present invention, reliable data can be obtained upon conducting a quality inspection, for example, regarding a method for identifying the types of beads, for example, on the basis of location information of probe arrays, for example.

### BRIEF DESCRIPTION OF THE DRAWINGS

FIG. 1 shows an example of the absorbance spectrum and the fluorescent spectrum of semiconductor nanoparticles.

FIG. 2 shows an illustration regarding a four-color stain of beads.

FIG. 3 shows an illustration regarding an example of the identification of the order of beads.

FIG. 4 shows an example of beads arranged two-dimensionally.

### DESCRIPTION OF THE PREFERRED EMBODIMENTS

In the following, an embodiment of the present invention is described specifically with reference to the drawings. The following description uses an example in which the arrangement or the order of beads that have embedded semiconductor nanoparticles as pigments is identified regarding the probe array technology. However, the present invention is not limited to this, but widely applied for identifying various materials. Although the example pertains to the identification of the beads arranged one-dimensionally, the same identification applies even if the arrangement of the beads is two-dimensional.

In the present invention, semiconductor nanoparticles are used for description, since the semiconductor nanoparticles are preferably coloring pigments, for example, used in the process of coloring the beads.

The semiconductor nanoparticles are characterized in that they emit narrow and strong fluorescence at the full width at half maximum (FWHM). Also, various fluorescent colors can be created, and future applications and uses are considered to cover extremely diverse types, so that the materials have attracted much attention.

Since semiconductor nanoparticles whose particle size is 10 nm or less are located in the transition region between bulk semiconductor crystals and molecules, their physicochemical properties are different from those of both bulk semiconductor crystals and molecules. In such a region, a quantum size effect is developed in which the degeneration of an energy band observed in bulk semiconductors is removed and orbits are dispersed, and the energy width of a forbidden band changes depending on particle size. Due to the development of the quantum size effect, the energy width of the forbidden band of the semiconductor nanoparticles decreases or increases in accordance with increases or decreases in particle size. The change of the energy width of the forbidden band has an influence on the fluorescence properties of the particles. If the particle size is small and the energy width of the forbidden band is wide, the fluorescence wavelengths are on the shorter wavelength side. If the particle size is large and the energy width of the forbidden band is small, the fluorescence wavelengths are on the longer wavelength side. In other words, semiconductor nanoparticles are attracting attention as materials capable of creating any fluorescent colors through control of particle size.

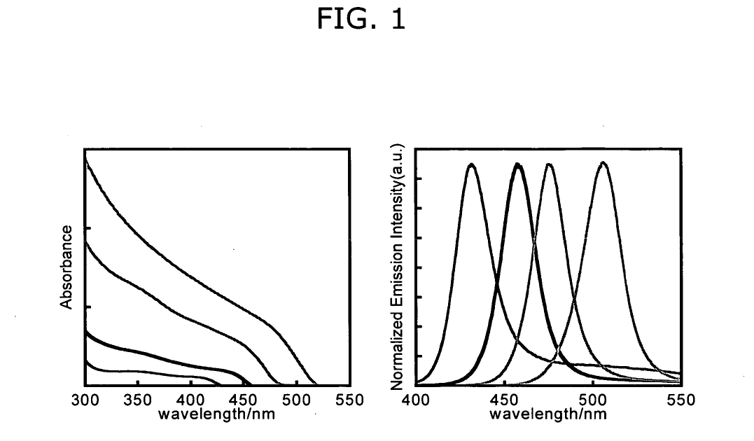

When synthesizing semiconductor nanoparticles that have properties of high emission of which the full width at half maximum is narrow, the control of the particle size and the modification of the particle surface are required. The inventors found that the semiconductor nanoparticles that have properties of high emission can be synthesized by conducting particle size control using a size-selective photoetching technique, and particle surface modification using sodium hydrate, amine compounds, ammonia compounds, and the like. By using this technology, the semiconductor nanoparticles that have properties of high emission in various fluorescence wavelengths as shown in FIG. 1 can be synthesized.

JP Patent Publication (Kokai) No. 11-243997 A (1999) discloses probe arrays in detail and JP Patent Publication (Kokai) No. 2000-346842 A and JP Patent Publication (Kokai) No. 2003-185663 A disclose manufacturing methods thereof. However, in the aforementioned technologies, the problem is that the order of the beads cannot be confirmed upon quality inspection, for example, and it is an object to construct an inspection method thereof.

Meanwhile, staining of the beads using nanoparticles is possible by applying methods for manufacturing conventional glass beads and polystyrene beads. U.S. Patent Application Publication No. 2003148544 and Nature Biotechnology (2001), 19(7), 631-635, for example, disclose specific methods for manufacturing stained beads using semiconductor nanoparticles. Fluorescence emitted by the semiconductor nanoparticles can be measured using commercially available flow cytometers, for example.

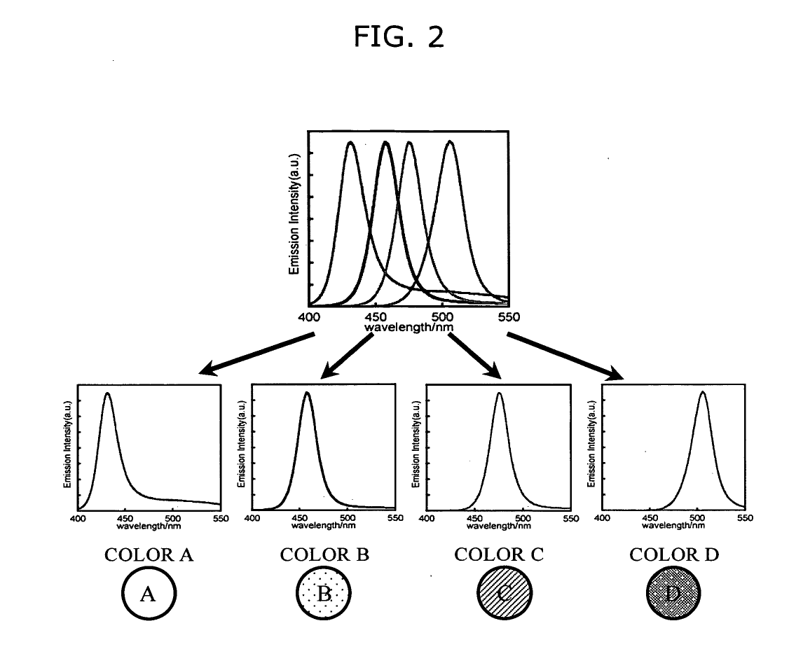

In the present invention, beads stained with individual colors are used. The number of stain colors for each bead can be one or a plurality of colors. In this case, a method for identifying the order of beads previously arranged in a capillary, for example, is described with reference to the drawings. The aforementioned arranged beads are stained using the semiconductor nanoparticles, and the types thereof have four colors (FIG. 1). Thus, the beads stained by the semiconductor nanoparticles have four types, which can result in beads stained with color A, color B, color C, and color D, as shown in FIG. 2.

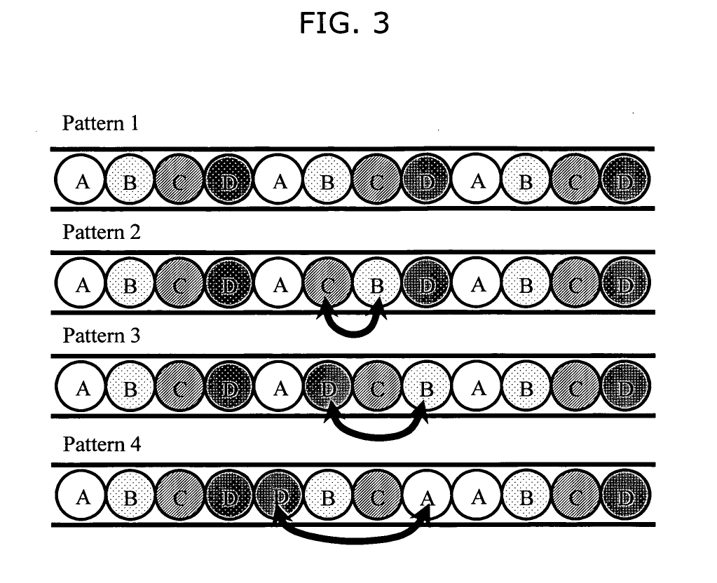

FIG. 3 shows an example of beads arranged one-dimensionally. Pattern 1 in FIG. 3 indicates a case where the beads are correctly arranged, in which an arrangement of A-B-C-D is repeated. By contrast, Patterns 2 to 4 indicate cases where the order is not correct. In the case of Pattern 2, the arrangement of A-B-C-D is developed partly and an error of order as suggested by the arrow in the figure can be detected in this case. In the same manner, in the cases of Patterns 3 and 4, an error of order as in the figure can be detected.

However, when the number of bead colors is represented by N, an error of order cannot be determined with respect to x+(N×n)th and x+N×(n+1)th (x: any integer). In this case, the arrangement is the same as in Pattern 1. In the same manner, an error of order with respect to x+(N×n)th and x+N×(n+1)+1th is the same as in Pattern 2, and errors of order with respect to x+(N×n)th and x+N×(n+1)+2th, and x+(N×n)th and x+N×(n+1)+3th are the same as in Patterns 3 and 4, respectively. In other words, the method for identifying the order according to the present invention is effective only within N from a base point, and a switch of the order to an extent greater than N cannot be detected. However, the detection system can be increased to an unlimited extent by securing a sufficient number for N.

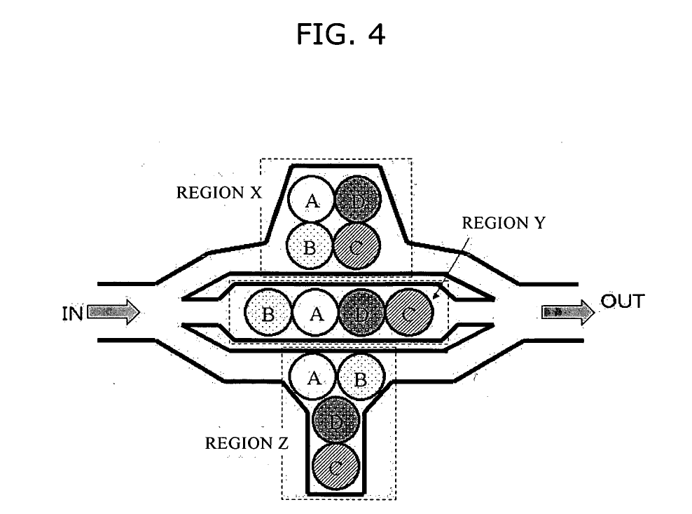

FIG. 4 shows an example of beads arranged two-dimensionally. A plurality of stained beads are bound in a selected form and in a selected region. In this case, the stained beads are identified in each selected form and selected region. Thus, the beads stained with the same color A are different types in bind field X, bind field Y, and bind field Z. Specific types of beads are specified by contrasting location information and color information in each bind field.

Although four colors (color A to color D) are used in this case, the number of colors is not limited as long as their wavelengths can be discriminated. For example, when semiconductor nanoparticles are used, the number of colors thereof can be in the tens of thousands, by embedding semiconductor nanoparticles that have two or more types of different color development in a single bead. Therefore, the identification method according to the present invention can be used for identification of extremely numerous types of items.

Reliable data can be obtained upon conducting a quality inspection, for example, using the method according to the present invention for identifying the types of beads, for example, on the basis of location information of probe arrays, for example.

1. A method for identifying the arrangement state of a plurality of beads on the basis of stain color information and location information concerning said plurality of types of stained beads.

2. The identification method according to claim 1, wherein said plurality of types of beads are arranged in a single line in the channel of a bead array, said location information is one-dimensional, and said arrangement state is one-dimensional.

3. The identification method according to claim 1, wherein said plurality of types of beads are immobilized and arranged in a planar manner, said location information is two-dimensional, and said arrangement state is two-dimensional.

4. The identification method according to claim 1, wherein said plurality of types of stained beads emit ultraviolet rays, visible light, or infrared rays.

5. The identification method according to claim 1, wherein said plurality of types of stained beads comprise beads that have embedded semiconductor nanoparticles, or beads that carry the semiconductor nanoparticles on the surface thereof, and said stain color comprises fluorescent colors.

6. The identification method according to claim 1, wherein said plurality of types of stained beads comprise beads that have embedded stain or pigment, or beads that carry the stain or the pigment on the surface thereof, and said stain color comprises the colors of visible light.

7. An identification method for a probe array technology relating to the detection and/or the measurement of biopolymers using a plurality of types of beads arranged in a capillary, said method employing the identification method for identifying the arrangement state of a plurality of beads on the basis of stain color information and location information concerning said plurality of types of stained beads in order to identify the order of beads arranged inside the capillary.
